(19) United States
(12) Patent Application Publication
(10) Pub. No.: US 2013/0034555 A1
(43) Pub. Date: Feb. 7, 2013

(54) EPITHELIAL MEMBRANE PROTEIN 2 (EMP2) BINDING REAGENTS AND THEIR THERAPEUTIC USES IN OCULAR DISEASES

(71) Applicants: Lynn K. Gordon, Tarzana, CA (US); Jonathan Braun, Tarzana, CA (US); Shawn A. Morales, Pasadena, CA (US); David Telander, Carmichael, CA (US); Madhuri Wadehra, Manhattan Beach, CA (US)

(72) Inventors: Lynn K. Gordon, Tarzana, CA (US); Jonathan Braun, Tarzana, CA (US); Shawn A. Morales, Pasadena, CA (US); David Telander, Carmichael, CA (US); Madhuri Wadehra, Manhattan Beach, CA (US)

(73) Assignees: United States Government Department of Veterans Affairs, Washington, DC (US); The Regents of the University of California, Oakland, CA (US)

(21) Appl. No.: 13/580,100

(22) PCT Filed: Feb. 22, 2011
(86) PCT No.: PCT/US2011/025772

§ 371 Date: Oct. 10, 2012

Related U.S. Application Data
(60) Provisional application No. 61/306,073, filed on Feb. 19, 2010.

(51) Int. Cl. A61K 39/395; A61P 27/02; A61K 31/7088

(52) U.S. Cl. 424/135.1; 424/172.1; 514/44 A

(57) ABSTRACT

A method is disclosed for treating diseases or disorders of the eye involving undesired expression of vascular endothelial growth factor (VEGF) by administration of endothelial membrane protein 2 (EMP2) inhibitor. The present invention relates to the direct treatment of macular degeneration, particularly age-related macular degeneration (AMD), by administering an EMP2 inhibitor intraocularly or systemically.

### CROSS-REFERENCES TO RELATED APPLICATIONS

The present application claims priority to provisional application U.S. Ser. No. 61/306,073, filed Feb. 19, 2010, herein incorporated by reference in its entirety.

### STATEMENT AS TO RIGHTS TO INVENTIONS MADE UNDER FEDERALLY SPONSORED RESEARCH OR DEVELOPMENT

This work was supported by the U.S. Department of Veterans Affairs, and the Federal Government has certain rights in this invention.

### BACKGROUND

In the industrialized world the average life expectancy is over 80 years of age and is increasing steadily. Unfortunately, the quality of life for the elderly is often dramatically decreased by the ocular condition known as age related macular degeneration (“ARMD” or “AMD”). The risk of the disease reaches more than 5% for individuals older than 80.

AMD is the leading cause of blindness in Americans over the age of 60 and the third leading cause of worldwide blindness. The World Health Organization has estimated that about 14 million people are blind or severely impaired because of AMD. The affliction of AMD has great impact on the physical and mental health of the geriatric population and their families and presents a significant public health care burden.

There are two forms of AMD, atrophic or dry AMD and neovascular or wet (exudative) AMD. Typically AMD begins as dry AMD. Dry AMD is characterized by the formation of yellow plaque like deposits called drusen in the macula, between the retinal pigment epithelium (RPE) and the underlying choroid. About 15% of dry AMD patients develop wet AMD, which leads to severe vision loss.

The severe vision loss associated with wet AMD is caused by the growth of abnormal new blood vessels from the choriocapillaris, a process call choroidal neovascularization (CNV). The new vessels tend to bleed, exude serum and promote excessive reparative responses within the macula. These changes, in turn, alter the anatomical relationship between the overlying neurosensory retina and the underlying RPE layer, causing detachment, dysfunction and degeneration of the photoreceptors. In the most severe cases, participants lose the ability to read or perform activities of daily living without aid.

AMD prophylaxis and therapy has changed radically over the past decade, beginning with the Age-Related Eye Disease Study, which in 2001 described a reduction in the risk for AMD progression of 25% in individuals who used vitamin supplements containing antioxidants and zinc.

Currently there are only four treatments approved by the FDA for wet AMD: laser surgery, photodynamic therapy (PDT), and the drugs Macugen®, pegaptanib sodium and Lucentis™ (ranibizumab) intravitreal injections. Laser, PDT and pegaptanib may slow the rate of vision decline and/or stop vision loss.

Laser surgery attempts to destroy the fragile, leaky blood vessels using a high energy beam of light. This treatment, however, may also destroy some surrounding healthy tissue and therefore actually contribute to further vision loss. Because of this, only a small percentage of people with wet AMD can be treated with laser surgery.

Photodynamic therapy also attempts to destroy the newly formed blood vessels in the patient's eye. Verteporfin (Visudyne®) is injected into the patient's arm. The drug travels through the patient's body, “sticking” to the surface of new blood vessels. A light is then shone in the patient's eye, which activates the drug, which in turn destroys the new blood vessel. Photodynamic therapy merely temporarily slows the rate of vision loss; it does not stop vision loss or restore vision. Moreover, because the drug is activated by light, the patient must avoid sunlight and bright indoor lights for five days after treatment.

The neovasculariztion associated with AMD was first effectively treated with photodynamic therapy, but actual improvements in vision were not achieved until the use of intraocular injections of anti-VEGF (vascular endothelial growth factor) reagents. Pegaptanib (Macugen®, Eyetech Pharmaceuticals Inc. and Pfizer Inc.), is approved for treatment of wet AMD is a pegylated oligonucleotide aptamer targeting VEGF. Ranibizumab (Lucentis™), an antibody fragment targeting VEGF, has recently been approved by FDA for the treatment of wet AMD.

Unfortunately, anti-VEGF therapy suffers from a significant drawback; the retina requires some levels of VEGF to be present for normal function. Studies have demonstated adverse affect to the retina due to low VEGF levels following anti-VEGF treatment. Thus, a need remains for methods and compositions which are useful in the treatment and/or prevention of AMD and other afflictions of the eye.

The 4-transmembrane (tetraspan) protein EMP2 (Epithelial Membrane Protein 2) is expressed at discrete locations in the eye. In the eye, EMP2 is localized to multiple epithelial layers including the cornea, ciliary boding, and RPE.

This invention provides novel methods for treating and/or preventing AMD and other affictions of the eye using reagents that bind to or regulate EMP2 protein or EMP2 nucleic acid.

### BRIEF SUMMARY OF THE INVENTION

The instant invention provides, in some embodiments, methods for treating neovascular diseases of the eye, specifically wet age related macular degeneration (AMD). The disclosed method entails the administration to a subject in need an effective amount of an epithelial membrane protein 2 (EMP2) inhibitor.

In some embodiments, the EMP2 inhibitor is an antibody. These antibodies are capable of specifically binding to EMP2. In addition, the disclosed anti-EMP2 antibodies can be polyclonal or monoclonal. Further, the anti-EMP2 antibodies may be a human antibody, a humanized anitbody, a chimeric antibody, a recominant antibody, a diabody, minibody, triabody, or an antibody fragment having the light and heavy chain variable sequences or CDRs corresponding to KS49, KS83, KS41, and KS89.

In some embodiments, the anti-EMP2 antibody encompasses a heavy chain and a light chain, wherein the heavy chain encompasses three complementarity determining regions (CDRs): HCDR1, HCDR2, and HCDR3, and wherein the light chain comprises three CDRs: LCDR1, LCDR2, and LCDR3, wherein HCDR1 encompasses SEQ ID NO:8, HCDR2 encompasses SEQ ID NO:9, and HCDR3 encompasses SEQ ID NO:10, and wherein LCDR1 encompasses SEQ ID NO:12, LCDR2 encompasses SEQ ID NO:13, and LCDR3 encompasses SEQ ID NO:14.

In other embodiments, the invention provides methods for treating AMD wherein the EMP2 inhibitor is an EMP2 si-RNA. In some embodiments, the EMP2 si-RNA encompasses a target sequence wherein the target sequence encompasses SEQ ID NO:3, SEQ ID NO:4, SEQ ID NO:5, or SEQ ID NO:6.

In other embodiments, the EMP2 si-RNA encompasses a target sequence selected from the group: SEQ ID NO:3, SEQ ID NO:4, SEQ ID NO:5, and SEQ ID NO:6.

In some embodiments, the EMP2 inhibitors of the invention are formulated into pharmaceutical compositions. These pharmaceutical compositions, in some embodiments, are suitable for adminstration to the eye. In some embodiments, the EMP2 inhibitor is administered to the eye by an intraocular route. In other embodiments, the EMP2 inhibitor is administered topically or subconjunctivally. These compositions are provided in an effective amount.

In some embodiments, methods are provided for treating diseases of the eye which include, for example, diabetic retinopathy, corneal neovascularization, choroidal neovascularization, cyclitis, Hippel-Lindau Disease, retinopathy of prematurity, pterygium, histoplasmosis, iris neovascularization, macular edema, glaucoma-associated neovascularization, and Purtscher's retinopathy.

In other embodiments, a method for reducing the expression of vascular endothelial growth factor (VEGF) in the eye is provided. The disclosed method provides for the adminstration of an effective amount of an epithelial membrane protein 2 (EMP2) inhibitor to a subject in need thereof. In some embodiments, the method for reducing the expression of VEGF in the eye entails the use of an anti-EMP2 antibody. In other embodiments, the EMP2 inhibitor is an EMP2 si-RNA.

In some embodiments, a method for treating wet AMD is provided, the method entailing the adminstration of an effective amount of at least two epithelial membrane protein 2 (EMP2) inhibitors to a subject in need thereof. In some embodiments, the two EMP2 inhibitors are an anti-EMP2 antibody and an EMP2 si-RNA.

### BRIEF DESCRIPTION OF THE DRAWINGS

FIG. 1 demonstrates that EMP2 over-expression increases VEGF expression. Cell extracts fractionated by 4-20% SDS-PAGE gradient gel in reducing conditions. (A) Representative immunoblots. (B) Band density, normalized to the β-actin loading control, was quantified.

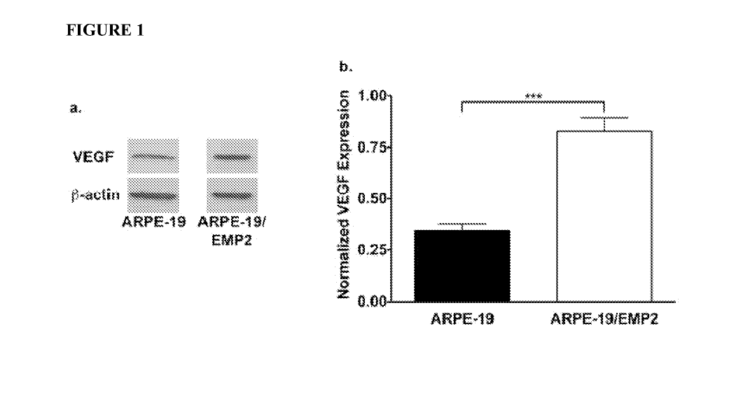

FIG. 2 demonstrates the effect of anti-EMP2 diabodies on VEGF expression. (A) Anti-EMP2 treatment significantly decreases VEGF levels (middle bar) as reveled by ELISA as compared to untreated (left bar) and control diabody (right bar). (B) Results of a Western blot analysis of VEGF expression following treatment with anti-EMP2 diabodies. Anti-EMP2 diabody treatment (right bar) significantly decreases VEGF levels as relative to untreated cells.

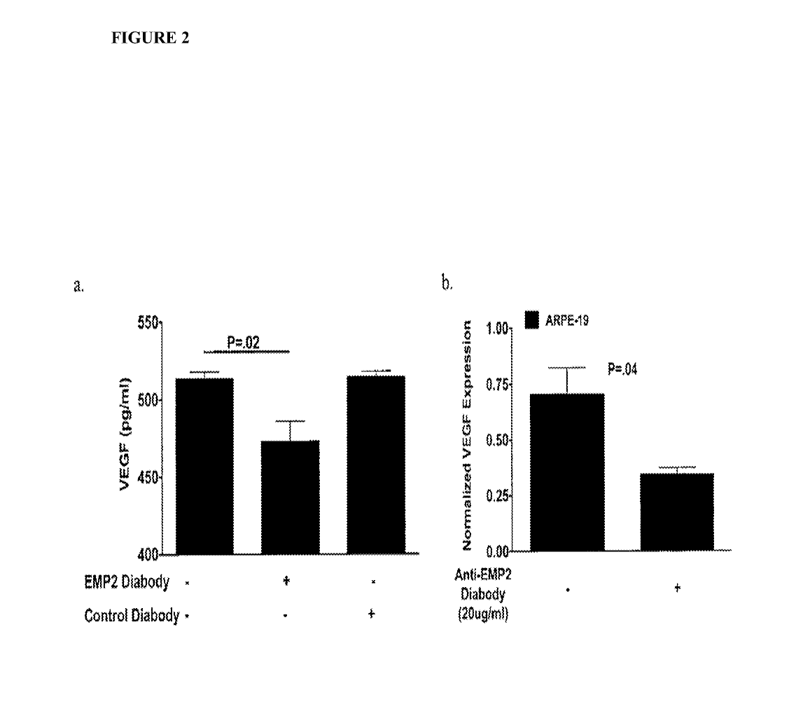

FIG. 3 demonstrates a time course of VEGF reduction following anti-EMP2 diabody treatment.

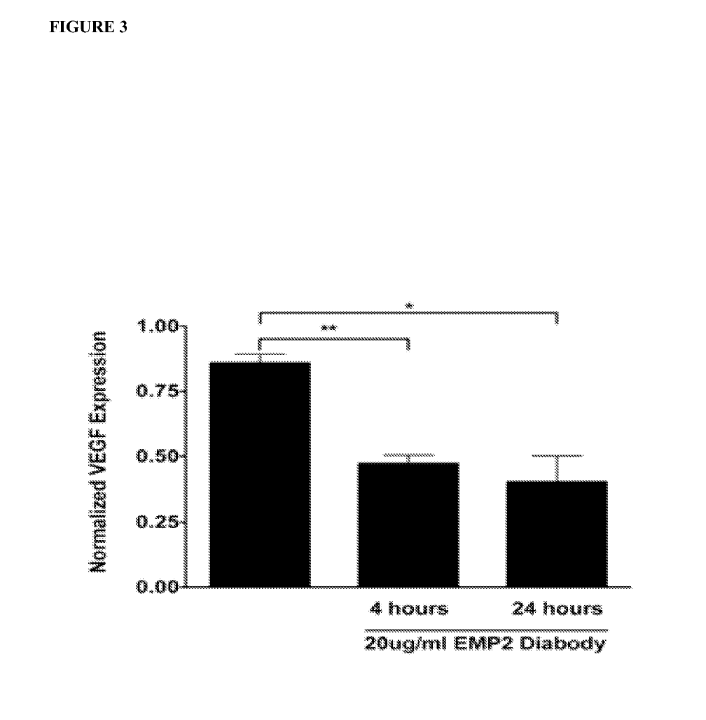

FIG. 4 demonstrates that si-RNA for EMP2 decreases VEGF expression. Cells treated with EMP2 specific si-RNA (middle bar) showed a significant decrease in VEGF expressio as compared to untreated cells (left bar) or cells treated with control si-RNA (right bar).

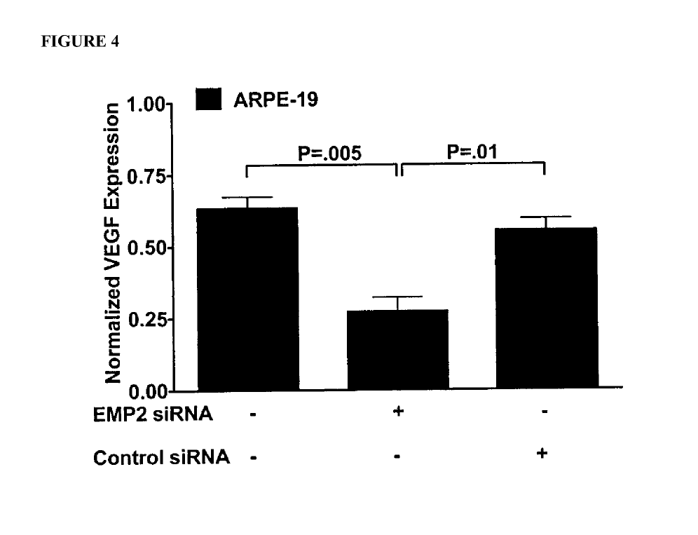

### DETAILED DESCRIPTION OF THE INVENTION

### Introduction

Age-related macular degenertion (“ARMD” or “AMD”) is a complex disease whose risk factors include aging, family history of AMD, smoking, hypertension, obesity, diet, and ethnicity, and there is a strong indication of a genetic contribution. Ambati et al., Surv. Ophalmol., 48:257 (2003). In the United States more than 50% of the cases of visual impairment occur secondary to a disease refered to as AMD. Two major clinical phenotypes of AMD are recognized: a nonexudative (thy) type and an exudative (wet) type. The most severe form of AMD, wet AMD results from pathological neovascularization of the retina called choroidal or subretinal neovasculariztion.

AMD is associated with changes to the retinal pigment epithelium (RPE). RPE is a monolayer of cuboidal, polarized epithelium with close contact to the neurosensory retina at the apical membrane. RPE is considered pivotal in maintaining retinal homeostasis and normal retinal functions. RPE is also responsible for the regulation of inflammatory response in the ocular microenvironment and for the maintaince of the blood brain barrier. It is believed that RPE is responsible for production of VEGF, a step in AMD pathogenesis.

EMP2 (epithelial membrane protein 2), which is relatively highly expressed in RPE, is a member of the growth arrest specific gene 3/peripheral mylein protein 22 (GAS3/PMP22) group of the tetraspan protein superfamily. EMP2 has at least two supposed biochemical roles in receptor-mediated cellular behavior. First, EMP2 regulates trafficking and intracellular compartmetalization and surface display of selected receptors and glycolipids. Second, EMP2 physically associates with and is thought to regulate the activity of integrin-FAK (focal adhesion kinase) signaling complexes.

The instant disclosure provides methods and compositions for treating ocular conditions and diseases using an effective amount of EMP2 inhibitors.

An “EMP2 inhibitor” refers to an agent that inhibits a function, activity, or levels of EMP2 using assays known in the art. An EMP2 inhibitor can be an EMP2 polypeptide; an anti-EMP2 antibody; an EMP2 siRNA molecule; an EMP2 aptamer; an EMP2-ribozyme; a compound which competes with binding of to EMP2, or an agent or compound which inhibits the expression, transcription, or translation of EMP2 nucleic acids in a host cell.

An EMP2 inhibitor can also reduce the function, activity or expression levels of VEGF. For example, an EMP2 inhibitor can reduce the presence of VEGF protein in a system by 2%, 5%, 10%, 15%, 20%, 25%, 30%, 35%, 40%, 45%, 50%, 55%, 60%, 65%, 70%, 75%, 80%, 85%, 90%, 95%, 98%, 99%, or more.

An “EMP2 polypeptide” refers to a conservatively modified variant of a polypeptide of SEQ ID NO: 1. Accordingly, in some embodiments, the EMP polypeptide consists of the sequence of EMP2 of SEQ ID NO:1 or a fragment thereof. The fragment may be from 15 to 25, 15 to 40, 25 to 50, 50 to 100 amino acids long, or longer. The fragment may correspond to that of EMP2 from position 16 to 64 of SEQ ID NO: 1.

A “polypeptide,” “peptide, and “protein” are used interchangeably and refer to a polymer of amino acid residues. Methods for obtaining (e.g., producing, isolating, purifying, synthesizing, and recombinantly manufacturing) polypeptides are well known to one of ordinary skill in the art.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g. , Creighton, Proteins (1984)).

### EMP2 Inhibitors

The present invention provides methods and reagents for inhibiting EMP2. Asays for identifying EMP2 inhibitors of the invention are conducted in the presence of the candidate inhibitor and then the results are compared to control samples without the inhibitor to examine for the desired activity or to determine the functional effect of the candidate inhibitor. A positive reference control, which is an agent having the desired activity, may be used.

The inhibition may take place in vivo or in vitro, and is accomplished by contacting a cell with a composition comprising an EMP2 inhibitor. Contacting in vitro may be accomplished, for example, by adding the EMP2 inhibitor to a cell culture medium. Contacting in vivo may be accomplished by administering a sterile or pharmaceutically acceptable composition with the EMP2 inhibitor to an animal (e.g., patient).

“Determining the functional effect” refers to assaying for a compound that increases or decreases a parameter that is indirectly or directly under the influence of a polynucleotide or polypeptide for use according to the invention, e.g., measuring physical and chemical or phenotypic effects.

Such functional effects can be measured by any means known to those skilled in the art, e.g., changes in spectroscopic (e.g., fluorescence, absorbance, refractive index), hydrodynamic (e.g., shape), chromatographic, or solubility properties for the protein; measuring inducible markers or transcriptional activation of the protein; measuring binding activity or binding assays, e.g. binding to antibodies; measuring changes in ligand binding affinity; e.g., via chemiluminescence, fluorescence, colorimetric reactions, antibody binding, inducible markers, and ligand binding assays.

A measure of EMP2 inhibition is a reduction in VEGF expression in cells contacted with an EMP2 inhibitor. Accordingly, inhibitors of EMP2 can be identified by contacting cells overexpressing EMP2 with an inhibitor and measuring a functional effect, for example VEGF expression levels. In some instances, such assays measure the presence of VEGF protein, for example by ELISA or Western blot. In other instances, such assays measure the presence of VEGF mRNA. Other suitable methods for measuring VEGF polypeptide and/or mRNA are known to those of skill in the art.

In some instances, when determining a functional effect, control samples can be assigned a relative of 100% Inhibition is achieved when the assayed function, activity or expression level is reduced by 2%, 5%, 10%, 15%, 20%, 25%, 30%, 35%, 40%, 45%, 50%, 55%, 60%, 65%, 70%, 75%, 80%, 85%, 90%, 95%, 98%, 99%, or more relative to the control.

Suitable methods for identifying inhibitors for use according to the invention are set forth in the Examples.

### Antibodies

In some embodiments, the EMP2 inhibitor is an antibody. An “anti-EMP2 antibody” or “EMP2 antibody” according to the invention is an antibody which can specifically bind to the EMP2 polypeptide of SEQ ID NO:1.

The antibodies for use according to the invention, but are not limited to, recombinant antibodies, polyclonal antibodies, monoclonal antibodies, chimeric antibodies, human monoclonal antibodies, humanized or primatized monoclonal antibodies, and antibody fragments. The antibodies preferably bind to an external loop sequence of EMP2.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody provides specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂ a dimer of Fab which itself is a light chain joined to V_{H}-C_{H}I by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993).

While various antibody fragments are defined in terms of the digestion of an intact antibody, one of skill will appreciate that such fragments may be synthesized de novo either chemically or by using recombinant DNA methodology. Thus, the term antibody, also includes antibody fragments either produced by the modification of whole antibodies, or those synthesized de novo using recombinant DNA methodologies (e.g., single chain Fv) or those identified using phage display libraries (see, for example, McCafferty et al., Nature 348:552-554 (1990)).

Accordingly, the term antibody also embraces minibodies, diabodies, triabodies and the like. “Diabodies” referes to small bivalent biospecific antibody fragments with high avidity and specificity. Their high signal to noise ratio is typically better due to a better specificity and fast blood clearance increasing their potential for diagnostic and therapeutic targeting of specific antigen (Sundaresan et al., J Nucl Med 44:1962-9 (2003).

In addition, these antibodies are advantageous because they can be engineered if necessary as different types of antibody fragments ranging from a small single chain Fv to an intact IgG with varying isoforms (Wu & Senter, Nat. Biotechnol. 23:1 137-1146 (2005)). In some embodiments, the antibody fragment is part of a diabody. In some embodiments, the invention provides high avidity antibodies for use according to the invention.

The following human-origin antibody sequences encode for high-avidity antibodies specific for human (KS49, KS83) and mouse (KS83) EMP2 and have antibody variable region heavy and light chains suitable for use in either aspect of the invention.

Anti-EMP-2 variable region sequences, used to encode proteins on backbones including for native antibody, fragment antibody, or synthetic backbones, can avidly bind EMP-2. Via this binding, these proteins can be used for EMP-2 detection, and to block EMP-2 function.

In some embodiments, the invention provides antibodies (e.g., diabodies, minibodies, triabodies) or fragments thereof having the CDRs of a diabody selected from KS49, KS83, KS41, and KS89. In some embodiments, these antibodies lack the polyhistine tag. In other embodiments, the diabodies possess the light and heavy chain of a KS49, KS83, KS41, or KS89 diabody. In still other embodiments, the antibodies are substantially identical in sequence to a diabody selected from the group consisting of KS49, KS83, KS41, and KS89 with or without the polyhistidine tag. In still other embodiments, the antibodies are substantially identical in sequence to the light and heavy chain sequences of a diabody selected from the group consisting of KS49, KS83, KS41, and KS89. These identities can be 65%, 70%, 75%, 80%, 85%, 90%, and preferably 91%, 92%, 93%, 94%, 95%, 96%, 97%, 98% or 99% or greater amino acid sequence identity. In some further embodiments of any of the above, the antibodies comprise CDRs sequences identical to those of the KS49, KS83, KS41, or KS89 diabody.

In some embodiments, the present invention provides anti-EMP-2 sequences comprising CDR regions of an antibody selected from KS49, KS83, KS41, and KS89. The CDR regions provided by the invention may be used to construct an anti-EMP-2 binding protein, including without limitation, an antibody, a scFv, a triabody, a diabody, a minibody, and the like.

In some embodiments, an anti-EMP-2 binding protein of the invention will comprise at least one CDR region from an antibody selected from KS49, KS83, KS41, and KS89. Anti-EMP-2 binding proteins may comprise, for example, a CDR-H1, a CDR-H2, a CDR-H3, a CDR-L1, a CDR-L2, a CDR-L3, or combinations thereof, from an antibody provided herein. In particular embodiments of the invention, an anti-EMP-2 binding protein may comprise all three CDR-H sequences of an antibody provided herein, all three CDR-L sequences of an antibody provided herein, or both.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologies.

Thus, under designated immunoassay conditions, the specified antibodies bind to a particular protein at least two times the background and more typically more than 10 to 100 times background. Specific binding to an antibody under such conditions requires an antibody that is selected for its specificity for a particular protein.

For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

For example, rabbit polyclonal antibodies to EMP2 are known in the art (see, Wang et al., Blood 97:3890-3895 (2001)). Such antibodies may be obtained using glutathione-S-transferase-EMP2 fusion proteins. Rabbit antibodies can be generated against the first extracellular region of the gene (from amino acid 16 to 64) constructed as a glutathione-5-transferase (GST)-EMP2 fusion protein. The EMP2 peptide can be cloned by PCR using the following primers:

CGCGGATCCTCTACCATTGACAATGCCTGG
(forward; BamH1 underlined);
CCGGAATTCTTACGCCTGCATCACAGAATAACC
(reverse, EcoR1 underlined).

The PCR product can be directionally cloned into the BamHI and EcoRI sites of the pGEX-4T-1 vector that contains GST gene (Pharmacia). The EMP2 fragment is cloned in frame with the GST to create a fusion protein. The insert can be confirmed by sequencing. The GST fusion protein can be produced as previously described (see, Smith D B et al., Gene 67:31-40 (1988)). Bacteria in log phase (OD₆₀₀ 0.6 to 0.9) can be induced for 2.5 to 3 hours at 37° C. with 1 mM isopropyl-l-thio-3-D-galactopyranoside. Bacteria are lysed, and the soluble fraction loaded onto a glutathione-Sepharose column (Pierce, Rockford, Ill.). The columns are washed with 10 bed volumes of phosphate-buffered saline (PBS)/EDTA. The fusion protein elutes from the column using 20 mM reduced glutathione (Sigma, St Louis, Mo.) in 50 mM Tris-Cl, pH 8.0. For antibody preparation, rabbits are immunized twice with the GST-EMP2 fusion protein, and serum is collected, starting 2 weeks after the last immunization (Research Genetics, Huntsville, Ala.).

Anti-EMP2 CDR sequences may be used on an antibody backbone, or fragment thereof, and likewise may include humanized antibodies, or antibodies containing humanized sequences. These antibodies may be used, for example, to detect EMP-2, to detect cells expressing EMP-2 in vivo, or to block EMP-2 function. In some embodiments, the CDR regions may be defined using the Kabat definition, the Chothia definition, the AbM definition, the contact definition, or any other suitable CDR numbering system.

Suitable methods for identifying antibody inhibitors for use according to the invention are set forth in Examples 4 and 6.

EMP2 si-RNA

In some embodiments, the EMP2 inhibitor is an EMP2 siRNA. An “siRNA” or “RNAi” refers to a nucleic acid that forms a double stranded RNA, which double stranded RNA has the ability to reduce or inhibit expression of a gene or target gene when the siRNA expressed in the same cell as the gene or target gene. “siRNA” or “RNAi” thus refers to the double stranded RNA formed by the complementary strands.

A method for evualating siRNA molecules for EMP2 inhibition can involve, for example, the addition of a candidate siRNA specific for EMP2 to test sample. Control samples can include, for example, samples in which no siRNA is added and/or samples in which irrelevant (not specific for EMP2) siRNA is added Inhibition of EMP2 or VEGF in the test sample relative to control sample(s) is indicative of a siRNA EMP2 inhibitor.

Suitable methods for identifying EMP2 siRNA inhibtors are provided in Examples 4, 5, and 6.

The complementary portions of the siRNA that hybridize to form the double stranded molecule typically have substantial or complete identity. In one embodiment, an siRNA refers to a nucleic acid that has substantial or complete identity to a target gene and forms a double stranded siRNA. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 or about 24-29 nucleotides in length, e.g., 20, 21, 22, 23, 24, 25, 26, 27, 28, 29, or 30 nucleotides in length.

The design and making of siRNA molecules and vectors are well known to those of ordinary skill in the art. For instance, an efficient process for designing a suitable siRNA is to start at the AUG start codon of the mRNA transcript and scan for AA dinucleotide sequences (see, Elbashir et al., EMBO J 20:6877-6888 (2001)). Each AA and the 3′ adjacent nucleotides are potential siRNA target sites. The length of the adjacent site sequence will determine the length of the siRNA. For instance, 19 adjacent sites would give a 21 Nucleotide long siRNA. This approach is also compatible with using RNA pol III to transcribe hairpin siRNAs. RNA pol III terminates transcription at 4-6 nucleotide poly(T) tracts to create RNA molecules having a short poly(U) tail. However, siRNAs with other 3′ terminal dinucleotide overhangs can also effectively induce RNAi and the sequence may be empirically selected. For selectivity, target sequences with more than 16-17 contiguous base pairs of homology to other coding sequences can be avoided by conducting a BLAST search (see, world wide web.ncbi.nlm.nih.gov/BLAST).

The siRNA can be administered directly or siRNA expression vectors can be used to induce RNAi. A vector can have inserted two inverted repeats separated by a short spacer sequence and ending with a string of T′s which serve to terminate transcription. The expressed RNA transcript is predicted to fold into a short hairpin siRNA . The selection of siRNA target sequence, the length of the inverted repeats that encode the stem of a putative hairpin, the order of the inverted repeats, the length and composition of the spacer sequence that encodes the loop of the hairpin, and the presence or absence of 5′-overhangs, can vary. A preferred order of the siRNA expression cassette is sense strand, short spacer, and antisense strand. Hairpin siRNAs with these various stem lengths (e.g., 15 to 30) are suitable. The length of the loops linking sense and antisense strands of the hairpin siRNA can have varying lengths (e.g., 3 to 9 nucleotides, or longer). The vectors may contain promoters and expression enhancers or other regulatory elements which are operably linked to the nucleotide sequence encoding the siRNA.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers. These control elements may be designed to allow the clinician to turn off or on the expression of the gene by adding or controlling external factors to which the regulatory elements are responsive.

In some embodiments, the EMP2 inhibitor is an EMP2 ribozyme which can inhibit the expression of EMP2 when present in a cell. Ribozymes are enzymatic RNA molecules capable of catalysing the specific cleavage of RNA. The mechanism of ribozyme action involves sequence specific hybridisation of the ribozyme molecule to complementary target RNA, followed by an endonucleolytic cleavage. Within the scope of the invention are engineered hammerhead motif ribozyme molecules that specifically and efficiently catalyse endonucleolytic cleavage of EMP2 mRNA. Specific ribozyme cleavage sites within any potential RNA target are initially identified by scanning the target molecule for ribozyme cleavage sites, which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable.

DNA molecules and ribozymes of the invention may be prepared by any method known in the art for the synthesis of RNA molecules. These include techniques for chemically synthesizing oligodeoxyribonucleotides well known in the art such as for example solid phase phosphoramidite chemical synthesis.

Alternatively, RNA molecules may be generated by in vitro and in vivo transcription of DNA sequences encoding the antisense RNA molecule. Such DNA sequences may be incorporated into a wide variety of vectors, which incorporate suitable RNA polymerase promoters such as the T7 or SP6 polymerase promoters. Methods of making ribozymes are well known in the art (see, for instance, U.S. Patent Application Publication No. 20060062785).

Construction of suitable vectors for the EMP2 siRNA or EMP2 Ribozymes containing the desired EMP2 siRNA or EMP2 Ribozyme sequences and control sequences employs standard ligation and restriction techniques, which are well understood in the art (see Maniatis et al., in Molecular Cloning: A Laboratory Manual, Cold Spring Harbor Laboratory, New York (1982)). Isolated plasmids, DNA sequences, or synthesized oligonucleotides are cleaved, tailored, and religated in the form desired.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine.

Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

The terms “identical” or percent “identity,” in the context of two or more nucleic acids or polypeptide sequences, including EMP2 siRNA and EMP2 polypeptides, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site hypertext transfer protocol://www.ncbi.nlm.nih.gov/BLAST/ or the like).

Such sequences are then said to be “substantially identical.” This also refers to, or may be applied to, the compliment of a test sequence. This also includes sequences that have deletions and/or additions, as well as those that have substitutions. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to the full length of the reference sequence, usually about 25 to 100, or 50 to about 150, more usually about 100 to about 150 in which a sequence may be compared to a reference sequence of the same number of contiguous positions after the two sequences are optimally aligned. Methods of alignment of sequences for comparison are well-known in the art.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. MoI. Biol. 48:443 (1970), by the search for similarity method of Pearson & Lipman, Proc. Nat 7. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection {see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. MoI. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (hypertext transfer protocol://www.ncbi.nlm nih.gov/). This algorithm involves first identifying high scoring sequence pairs (HSPs) by identifying short words of length W in the query sequence, which either match or satisfy some positive- valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., MoI. Cell. Probes 8:91-98 (1994)). The term nucleic acid is used interchangeably with gene, cDNA, mRNA, oligonucleotide, and polynucleotide.

“Heterologous” refers to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

### EMP2 Aptamers

In some embodiments, the EMP2 inhibitors are aptamers. Aptamers are nucleic acid molecules having specific binding affinity to molecules through interactions other than classic Watson-Crick base pairing.

Aptamers, like peptides generated by phage display or monoclonal antibodies are capable of specifically binding to selected targets and modulating the target's activity or binding interactions, e.g., through binding aptamers may block their target's ability to function. Discovered by an in vitro selection process from pools of random sequence oligonucleotides, aptamers have been generated for over 130 proteins including growth factors, transcription factors, enzymes, immunoglobulins, and receptors. A typical aptamer is 10-15 kDa in size (20-45 nucleotides), binds its target with nanomolar to sub-nanomolar affinity, and discriminates against closely related targets (e.g., aptamers will typically not bind other proteins from the same gene family).

A series of structural studies have shown that aptamers are capable of using the same types of binding interactions (e.g., hydrogen bonding, electrostatic complementarities, hydrophobic contacts, steric exclusion) that drive affinity and specificity in antibody-antigen complexes.

A suitable method for generating an aptamer is with the process entitled “Systematic Evolution of Ligands by Exponential Enrichment” (“SELEX™”). The SELEX™ process is a method for the in vitro evolution of nucleic acid molecules with highly specific binding to target molecules and is described in, e.g., U.S. patent application Ser. No. 07/536,428, filed Jun. 11, 1990, now abandoned, U.S. Pat. No. 5,475,096 entitled “Nucleic Acid Ligands”, and U.S. Pat. No. 5,270,163 (see also WO 91/19813) entitled “Nucleic Acid Ligands”.

SELEX™ relies as a starting point upon a large library or pool of single stranded oligonucleotides comprising randomized sequences. The oligonucleotides can be modified or unmodified DNA, RNA, or DNA/RNA hybrids. In some examples, the pool comprises 100% random or partially random oligonucleotides. In other examples, the pool comprises random or partially random oligonucleotides containing at least one fixed sequence and/or conserved sequence incorporated within randomized sequence. In other examples, the pool comprises random or partially random oligonucleotides containing at least one fixed and/or conserved sequence at its 5′ and/or 3′ end which may comprise a sequence shared by all the molecules of the oligonucleotide pool.

More specifically, starting with a mixture containing the starting pool of nucleic acids, the SELEX™ method includes steps of: (a) contacting the mixture with the target under conditions favorable for binding; (b) partitioning unbound nucleic acids from those nucleic acids which have bound specifically to target molecules; (c) dissociating the nucleic acid-target complexes; (d) amplifying the nucleic acids dissociated from the nucleic acid-target complexes to yield a ligand-enriched mixture of nucleic acids; and (e) reiterating the steps of binding, partitioning, dissociating and amplifying through as many cycles as desired to yield highly specific, high affinity nucleic acid ligands to the target molecule. In those instances where RNA aptamers are being selected, the SELEX™ method further comprises the steps of: (i) reverse transcribing the nucleic acids dissociated from the nucleic acid-target complexes before amplification in step (d); and (ii) transcribing the amplified nucleic acids from step (d) before restarting the process.

Within a nucleic acid mixture containing a large number of possible sequences and structures, there is a wide range of binding affinities for a given target. A nucleic acid mixture comprising, for example, a 20 nucleotide randomized segment can have 4²⁰ candidate possibilities. Those which have the higher affinity (lower dissociation constants) for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands or aptamers.

A method for evualating aptamers for EMP2 inhibition can involve, for example, the addition of a candidate aptamer to a test sample. Control samples can include, for example, samples in which no aptamer is added and/or samples in which irrelavant (not specific for EMP2) aptamer is added. Inhibition of EMP2 or VEGF in the test sample relative to control sample(s) is indicative of an aptamer EMP2 inhibitor.

### Small Molecules

In some embodiments, the EMP2 inhibitor is a small molecule. “Small molecule” refers to a compound, for example an organic compound, with a molecular weight of in one embodiment less than about 2,500 daltons, in another embodiment less than about 2000 daltons, in another embodiment less than about 1000 daltons, and in still another embodiment less than about 500 daltons.

Small molecules can be any small chemical compound, or a biological entity, such as a protein, sugar, nucleic acid or lipid.

Small molecules may be obtained from a wide variety of sources including libraries of synthetic or natural compounds.

In one embodiment, screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan. 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Small molecules that inhibit EMP2 may be identified and/or assessed in any number of ways. For example, an antagonist of EMP2 may be capable of inhibiting the production of VEGF, or interfere with the biological effects of VEGF. EMP2 inhibitors may also inhibit EMP2 signaling. Small molecule inhibitors of EMP2 may be identified using assays wherein the presumptive EMP2 inhibitor is added to a sample. Comparison is then made between those samples in which the presumptive small molecule inhibitor of EMP2 is added to controls.

### Ocular Conditions and Diseases

An “ocular condition” can include a disease or aliment that affects or involves the eye or one of the parts or regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball.

A “front of the eye” or “anterior ocular condition” is a disease or ailment that affects or which involves an ocular region or site, such as a periocular muscle, an eye lid or an eye ball tissue or fluid which is located anterior to the posterior wall of the lens capsule or ciliary muscles. Thus, a front of the eye ocular condition affects or involves, the conjunctiva, the cornea, the conjunctiva, the anterior chamber, the iris, the lens and the lens capsule as well as blood vessels, lymphatics and nerves which vascularize, maintain or innervate an anterior ocular region or site, or the like.

A front of the eye (anterior) ocular condition can include a disease, ailment or condition, such as for example, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can be considered to be a front of the eye ocular condition because a clinical goal of glaucoma treatment can be to reduce a hypertension of aqueous fluid in the anterior chamber of the eye (i.e. reduce intraocular pressure).

A “posterior ocular condition” (or back of the eye) is a disease or ailment that primarily affects or involves a posterior ocular region or site such as, for example, choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site or the like.

Thus, a posterior ocular condition can include a disease or ailment, such as, for example, macular degeneration (such as non-exudative age related macular degeneration and exudative age related macular degeneration); choroidal or retinal neovascularization. Glaucoma can also be considered a back of the eye ocular condition because of the damage to the optic nerve.

An “intraocular neovascular disease” is a disease characterized by ocular neovascularization. Examples of intraocular neovascular diseases include, but are not limited to, e.g., proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, corneal neovascularization, retinal neovascularization.

“Dry AMD” (also referred to as atrophic age related macular degeneration) refers to a retinal condition in which drusen are present in the macula but with little or no retinal neovascularization.

The dry form of AMD is associated with cell death of the light-sensitive macular part of the retina, which is required for fine vision used in activities such as reading, driving or recognizing faces. Over time, as less of the macula functions, central vision in the affected eye can be lost gradually. One of the most common early signs of dry AMD is the appearance of drusen.

Drusen are yellow deposits under the retina and are often found in people over the age of 60.

Early and intermediate AMD are characterized by the presence of small or medium-sized drusen, and persons suffering from early and intermediate AMD may require additional light when reading and experience a blurred spot in the center of their vision. Persons suffering from advanced AMD, in addition to the presence of medium or large-sized drusen, exhibit a breakdown of light-sensitive cells and supporting tissue in the central retinal area.

“Wet AMD” refers to a retinal condition characterized by the presence of retinal neovascularization (category 4 or advanced AMD) or vision loss.

Posterior ocular conditions also can include acute macular neuroretinopathy; macular edema (such as cystoid macular edema and diabetic macular edema); Behcet's disease, retinal disorders, diabetic retinopathy (including proliferative diabetic retinopathy); retinal arterial occlusive disease; central retinal vein occlusion; uveitic retinal disease; retinal detachment; ocular trauma which affects a posterior ocular site or location; a posterior ocular condition caused by or influenced by an ocular laser treatment; posterior ocular conditions caused by or influenced by a photodynamic therapy; photocoagulation; radiation retinopathy; epiretinal membrane disorders; branch retinal vein occlusion; anterior ischemic optic neuropathy; non-retinopathy diabetic retinal dysfunction, retinitis pigmentosa and glaucoma.

Glaucoma can also be considered a posterior ocular condition because a therapeutic goal of glaucoma treatment is to prevent the loss of or reduce the occurrence of loss of vision due to damage to or loss of retinal cells or optic nerve cells (i.e. neuroprotection).

In various embodiments, the ocular disease or condition treated will be wet (exudative) macular degeneration. “Treatment” and “therapy” may be used interchangeably. In certain embodiments, the therapy can be selected from, without limitation, initiating therapy, continuing therapy, modifying therapy or ending therapy. A therapy also includes any prophylactic measures that may be taken to prevent disease.

In certain embodiments, treatment comprises administering a disease-modulating drug to a subject. The drug can be a therapeutic or prophylactic used in subjects diagnosed or identified with a disease or at risk of having the disease. In certain embodiments, modifying therapy refers to altering the duration, frequency or intensity of therapy, for example, altering dosage levels.

An “effective amount” or “therapeutically effective amount” refers to the amount of a compound or drug necessary to prevent, amerliorate or treat a condition or disease. In the case of age-related macular degeneration (AMD), the effective amount of the drug can, for example, be the amount necessary to reduce or prevent vision loss.

For AMD therapy, efficacy in vivo can, for example, be measured by one or more of the following: assessing the mean change in the best corrected visual acuity (BCVA) from baseline to a desired time, assessing the proportion of subjects who lose fewer than 15 letters in visual acuity at a desired time compared with baseline, assessing the proportion of subjects who gain greater than or equal to 15 letters in visual acuity at a desired time compared with baseline, assessing the proportion of subjects with a visual-acuity Snellen equivalent of 20/2000 or worse at desired time, assessing the National Eye Institute Visual Functioning Questionnaire, assessing the size of CNV and amount of leakage of CNV at a desired time, as assessed by fluorescein angiography.

“Therapeutic dose” refers is a dose which exhibits a therapeutic effect on the patient and a sub-therapeutic dose is a dose which does not exhibit a therapeutic effect on the patient treated.

### Epithelial Membrane Protein (EMP) 2

Epithelial membrane protein 2 (EMP2)(polypeptide; SEQ ID NO: 1) (nucleic acid; SEQ ID NO: 2) is a member of the growth arrest-specific gene 3/peripheral myelin protein 22 four-transmembrane protein family with distinctive biochemical and physiologic roles. EMP2 appears to regulate trafficking of various proteins and glycolipids by facilitating transfer of molecules from post-Golgi endosomal compartments to appropriate plasma membrane locations.

Specifically, EMP2 is thought to facilitate the appropriate trafficking of select molecules into glycolipid-enriched lipid raft microdomains. Glycolipid-enriched microdomains are cholesterol-rich microdomains, which are often associated with chaperones, receptosomes, and protein complexes that are important for efficient signal transduction.

Moreover, glycolipid-enriched microdomains are involved in correct sorting of proteins from the Golgi apparatus to plasma membrane. In this respect, modulation of EMP2 expression levels or its location on the plasma membrane alters the surface repertoire of several classes of molecules including integrins, focal adhesion kinase (FAK), class I MHC molecules, and other immunoglobulin superfamily members such as CD54 and glycosylphosphatidylinositol-linked proteins.

As described above, in the eye EMP2 is found in the cornea, ciliary boding, and RPE.

### Vascular Endothelial Growth Factor (VEGF)

VEGF is expressed in a variety of cells in the normal human retina. Co-localization of VEGF mRNA and protein is observed in the ganglion cell, inner nuclear and outer plexiform layers, the walls of the blood vessels, and photoreceptors (Gerhardinger et al., Am J Pathol 152:1453-62 (1998)). Retinal pigment epithelium, Muller cells, pericytes, vascular endothelium, and ganglion cells all produce VEGF (Miller et al., Diabetes Metab Rev 13:37-50 (1997); and, Kim et al. Invest Opthalmol Vis Sci 40:2115-21 (1999)).

Immunohistochemical studies have localized VEGF in surgically resected CNV membranes from AMD patients. Kvanta et al. (1996) described the presence of VEGF mRNA and protein in RPE cells and fibroblast like cells. See Kvanta et al., Invest Opthalmol Vis Sci 37:1929-34 (1996). Lopez et al. (1996) noted that the RPE cells that were strongly immunoreactive for VEGF were present primarily in the highly vascularized regions of CNV membranes, whereas the RPE cells found in fibrotic regions of CNV membranes showed little VEGF reactivity. See Lopez et al., Invest Opthalmol V is Sci 37:855-68 (1996). Kliffen et al. (1997) described increased VEGF expression in RPE cells and choroidal blood vessels in maculae from patients with wet AMD compared with controls. See Kliffen et al., Br J Opthalmol 81:154-62 (1997).

An increase in VEGF expression has been noted in experimental models of CNV in rats and in non human primates (Husain et al., Opthalmology 104:124250 (1997); and, Yi et al. Vascular endothelial growth factor expression in choroidal neovascularization in rats. Graefes Arch Clin Exp Opthalmol 235:313-9 (1997)). In addition, transgenic mice with increased VEGF expression in photoreceptors (Okamoto et al. 1997, supra) or retinal pigment epithelium (Schwesinger et al., AM J Pathol. 158(3):1161-72 (2001)) developed neovacularization reminiscent of CNV seen in humans with neovascular AMD.

Of interest to wet AMD are the angiogenic properties of VEGF, which are described in a variety of in vivo models, including the chick chorioallantoic membrane (Leung et al., Science 246:1306-9 (1989); and, Plouet J, Schilling J, Gospodarowicz D. EMBO J 8:3801-6 (1989)), rabbit cornea (Phillips et al., In Vivo 8:961-5 (1994)), and rabbit bone (Connolly et al. J Clin Invest 84:1470-8 (1989a)). VEGF also potentially functions as a survival factor for newly formed endothelial cells (Dvorak H F. N Engl J Med 315:1650-9 (1986); and, Connolly et al. J Biol Chem 264:20017-24 (1989)). Consistent with pro survival activity, VEGF induces expression of the anti apoptotic proteins Bel 2 and Al in human endothelial cells (Connolly et al. J Biol Chem 264:20017-24 (1989b)).

VEGF has been shown to induce vascular leakage in guinea pig skin. A function of VEGF in the angiogenic process could be the induction of plasma protein leakage. This effect would result in the formation of an extravascular fibrin gel, which serves as a substrate for endothelial cells. Permeability of the CNV membranes could result in transudation of serum components beneath and into the retina, leading to serous macular detachment, macular edema and vision loss.

It has been shown that EMP2 regulates Focal Adhesion Kinase (FAK). FAK signal transduction is implicated in VEGF production.

### Pharmaceutical Compositions

Therapeutic compounds used in accordance with the present invention are prepared for storage by mixing a polypeptide(s) having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. In some embodiments, an intraocular implant can be used for providing the EMP2 inhibitor. Examples of sustained-release preparations include semipermeable matrices of solid hydrophobic polymers containing a polypeptide of the invention, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

Within the scope of the invention are suspensions of microspheres (incorporating an agent) suspended in a hydrogel (such as a polymeric hyaluronic acid) which can be administered to an intraocular location through a syringe needle.

The drug delivery systems of the invention can include a therapeutic agent mixed with or dispersed within a biodegradable polymer. The drug delivery systems compositions can vary according to the preferred drug release profile, the particular active agent used, the ocular condition being treated, and the medical history of the patient.

An intraocular drug delivery system can be made of a biodegradable polymer such as a poly(lactide) (PLA) polymers, poly(lactide-co-glycolide) (PLGA) polymers, as well as copolymers of PLA and PLGA polymers. PLA and PLGA polymers degrade by hydrolysis, and the degradation products, lactic acid and glycolic acid, are metabolized into carbon dioxide and water.

When encapsulated antibodies remain in the body for a long time, they may denature or aggregate as a result of exposure to moisture at 37° C., resulting in a loss of biological activity and possible changes in immunogenicity.

Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

### Administrative Modalities

The present invention also encompasses particular drug delivery system formulations and methods for administering these drug delivery systems for treating an ocular condition, such as AMD.

The therapeutic compound for treatment of an intraocular neovascular disease is typically administered by ocular, intraocular, and/or intravitreal injection. Other methods administration can also be used, which includes but is not limited to, topical, parenteral, for example, subcutaneous, intraperitoneal, intrapulmonary, intranasal, and intralesional administration.

Parenteral infusions include intramuscular, intravenous, intraarterial, intraperitoneal, or subcutaneous administration. As described herein, the therapeutic compound for treatment of an intraocular neovascular syndrome may be formulated, dosed, and administered in a fashion consistent with good medical practice.

“Intraocular” refers to within or under an ocular tissue. An intraocular administration of a drug delivery system includes administration of the drug delivery system to a sub-Tenon, subconjunctival, suprachoroidal, intravitreal and like locations. An intraocular administration of a drug delivery system excludes administration of the drug delivery system to a topical, systemic, intramuscular, subcutaneous, intraperitoneal, and the like location.

A “therapeutic agent,” “active agent,” and “drug” are used interchangeably and refer to any substance (including a biologic or macromolecule) used to treat an ocular condition. A therapeutic agent can be administered to the eye to treat an ocular condition. It is known to administer a drug depot to the posterior (i.e. near the macula) sub-Tenon space. See eg column 4 of U.S. Pat. No. 6,413,245. Additionally, it is known to administer a polylactic implant to the sub-tenon space or to a suprachoroidal location. See eg published U.S. Pat. No. 5,264,188 and published U.S. patent application 20050244463

An anti-EMP2 agent can be used for the treatment of an ocular condition, such as a posterior ocular condition, which involves angiogenesis such as choroidal neovascularization (“CNV”). A therapeutic amount of an anti-EMP2 agent can be administered to the eye directly, for example into the vitreous. Maurice, D. M. (1983) Micropharmaceutics of the eye, Ocular Inflammation Ther. 1:97-102; Lee, V. H. L. et al. (1989), Drug delivery to the posterior segment” Chapter 25 In Retina. T. E. Ogden and A. P. Schachat eds., St. Louis: C V Mosby, Vol. 1, pp. 483-98; and Olsen, T. W. et al. (1995), Human scleral permeablilty: effects of age, cryotherapy, transscleral diode laser, and surgical thinning, Invest. Ophthalmol. Vis. Sci. 36:1893-1903.

Intraocular administration can be injected to an intraocular location by syringe or can be inserted (implanted) into the eye by a variety of methods, including placement by forceps, by trocar, or by other types of applicators, after making an incision in the sclera.

In some instances, a trocar or applicator may be used without creating an incision. In some embodiments, a hand held applicator is used to insert one or more biodegradable implants into the eye. The hand held applicator typically comprises an 18-30 GA stainless steel needle, a lever, an actuator, and a plunger. Suitable devices for inserting an implant or implants into a posterior ocular region or site includes those disclosed in U.S. patent application Ser. No. 10/666,872.

The method of administration generally first involves accessing the target area within the ocular region with the needle, trocar or implantation device. Once within the target area, e.g., the vitreous cavity, a lever on a hand held device can be depressed to cause an actuator to drive a plunger forward. As the plunger moves forward, it can push the implant or implants into the target area (i.e. the vitreous by implantation or injection into the vitreous cavity (posterior chamber) of the eye.

The drug delivery systems can be biodegradable implants and/or microspheres. The drug delivery systems can be monolithic, that is the active agent is homogenously distributed or dispersed throughout the biodegradable polymer.

EP 488 401 discusses intraocular implants, made of certain polylactic acids, to be applied to the interior of the eye after a surgical operation for disorders of the retina/vitreous body. EP 430539 discusses use of a bioerodible implant which is inserted in the suprachoroid. U.S. application Ser. No. 11/565,917 filed Dec. 1, 2006 discloses intraocular (including sub-tenon's) administration of various solid, drug-containing implants.

Intraocular drug delivery systems which are sutured or fixed in place are known. Suturing or other fixation means requires sensitive ocular tissues to be in contact with aspects of a drug delivery system which are not required in order to contain a therapeutic agent within or on the drug delivery system or to permit the therapeutic agent to be released in vivo. As such suturing or eye fixation means a merely peripheral or ancillary value and their use can increase healing time, patient discomfort and the risk of infection or other complications.

U.S. patent applications Ser. Nos. 11/742,350; 11/859,310; 11/952,938; 11/364,687 discuss use of intraocular compositions comprising therapeutic antibodies, such as bevacizumab. Formulations of macromolecules for intraocular use are known, See eg applications Ser. Nos. 11/370,301; 11/364,687; 60/721,600; 11/116,698 and 60/567,423.

### Treatment Modalities

The doses may be administered according to any time schedule which is appropriate for treatment of the disease or condition. For example, the dosages may be administered on a daily, weekly, biweekly or monthly basis in order to achieve the desired therapeutic effect and reduction in adverse effects.

The dosages can be administered before, during or after the development of the disorder. The specific time schedule can be readily determined by a physician having ordinary skill in administering the therapeutic compound by routine adjustments of the dosing schedule within the method of the present invention.

The time of administration of the number of first individual and second individual doses as well as subsequent dosages is adjusted to minimize adverse effects while maintaining a maximum therapeutic effect. The occurrence of adverse effects can be monitored by routine patient interviews and adjusted to minimize the occurrence of side effects by adjusting the time of the dosing. Any dosing time is to be considered to be within the scope of the present invention. For example, doses may be administered on a monthly schedule followed by subsequent quarterly or more dose schedule. Maintenance doses are also contemplated by the invention.

The dosage amount depends on the specific disease or condition which is treated and can be readily determined using known dosage adjustment techniques by a physician having ordinary skill in treatment of the disease or condition. The dosage amount will generally lie with an established therapeutic window for the therapeutic compound which will provide a therapeutic effect while minimizing additional morbidity and mortality. Typically, therapeutic compounds are administered in a dosage ranging from 0.001 mg to about 100 mg per dose, preferably 0.1-20 mg.

Typically, the therapeutic compound used in the methods of this invention is formulated by mixing it at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of antagonist, but preferably ranges anywhere from about 3 to about 8.

The therapeutic compound, for example an anti-EMP2 antibody, for use herein is sterile. Sterility can be readily accomplished by sterile filtration through (0.2 micron) membranes. Preferably, therapeutic peptides and proteins are stored as aqueous solutions, although lyophilized formulations for reconstitution are acceptable.

The therapeutic compound may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the time scheduling of administration, and other factors known to medical practitioners.

It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like. The conjunction “or” is not mutually exclusive, unless context clearly dictates otherwise. The term “include” is used to refer to non-limiting examples.

### EXAMPLES

The following examples are offered to illustrate, but not to limit the invention.

### Example 1

### ARPE-19 Cell Line

ARPE-19, a spontaeously arising retinal pigment epithelial (RPE) cell line that expresses the RPE-specific markers CRALBP and RPE-65, was obtained from the American Type Culture Colletion (CRL-2302; ATCC™) ARPE-19 cells were cultured in DMEM-F12 medium, supplemented with 10% fetal bovine serum at 37° C. in a humidified chamber with 5% CO₂. The culture medium was replaced twice a week. After confluence, the cultures were passaged by dissociation in 0.05% (wt/vol) trypsin. Levels of EMP2 were increased.

### Example 2

### Phage Library Selection

10¹² to 10¹³ phage from the 8.2×10⁸ member phagemid library were first predepleted with 100 μL streptavidin magnetic beads (Invitrogen) in 2% milk PBS for 1 h at room temperature. The predepleted phage library was then mixed with 10 ng biotin-conjugated 24-amino acid peptides corresponding to the extracellular loop of hEMP2 and mouse EMP2 (mEMP2; DIHDKNAKFYPVTREGSYGGSGSK and DLHQQNRKLYYLLQEGSYGGSGSK, respectively; ref. 27) for 1 h at room temperature. One hundred microliters of 2% milk PBS preblocked streptavidin magnetic beads were added to the phage mixture and rotated for 15 min at room temperature. Beads were washed extensively with 0.1% PBS/Tween 20, 2% milk PBS, and finally with PBS, and bound phage was eluted out with 1 mL of 100 mmol/L triethylamine, neutralized with 500 μL of 1 mol/L Tris-HCl (pH 7.4), and added to 10 mL exponentially growing Escherichia coli TG1. Culture was then plated on 150 mm culture plates with 2×TY 100 μg/mL ampicillin and 2% glucose agar plates overnight at 37° C. The next day, colonies were scraped from the plates and used to amplify the phage for the second round of selection described above. A total of three selections were done before screening and characterization of the selected phage antibodies.

### Example 3

### Diabody Construction and Production

Binding specificity of expressed scFv was analyzed by ELISA. scFv clones with high reactivity were selected for the construction of diabodies. Several different scFv clones were characterized and confirmed by DNA fingerprinting and DNA sequencing. pHEN phagemids from selected phage were isolated using QIAprep Spin Miniprep Kit (Qiagen). scFv inserts were then digested and cloned into pSYN I vector in-frame with a c-Myc and 6 His tag at the COOH terminus To convert scFv fragments into diabody, 15-amino acid linker region (AGTGGTGGAGGCGGTTCAGGCGGAGGTGGCTCTGGCGGTGGCGGATCG) of the scFv was shortened into 5-amino acid linker (AGTGGTGGAGGATCG) using QuikChange site-directed mutagenesis kit (Stratagene). Deletion mutation was confirmed by DNA sequencing analysis.

Expression and purification of the selected diabodies were carried out using a modified protocol described by Marks and Bradbury. Single colonies were picked from the plate and inoculated into 1 L/colony of 2×TY with 100 μg/mL ampicillin at 250 rpm at 37° C. When A₆₀₀ reached 0.8 to 1.0, protein expression was induced by addition of 1 mmol/L IPTG. The culture was shaken at 120 rpm at 30° C. for 4 h and spun at 7,000 rpm for 15 min at 4° C. Pellets were then resuspended in 20 mL periplasmic buffer [200 mmol/L Tris-HCl, 20% sucrose, 1 mmol/L EDTA (pH 7.5)], and 290,000 units lysozyme (Epicentre) was added to each mixture. The mixtures were incubated at room temperature for 5 min and spun at 7,000 rpm for 15 min at 4° C. The pellets were then resuspended with 20 mL of 40 mmol/L MgSO₄ and left on the ice for 10 min. The samples were spun again, and the supernatants from this spin were combined with the first supernatants. The mixture was then filtered with 0.45 μm filters and dialyzed in dialysis buffer [300 mmol/L NaCl, 20 mmol/L HEPES (pH 8.0)] overnight at 4° C. Next morning, the samples were filtered again with 0.2 μm filters and run through 5 mL of the Ni-NTA column (Qiagen). The column was washed with 20 mL wash buffer [300 mmol/L NaCl, 20 mmol/L imidazole, 20 mmol/L HEPES, 0.05% Tween 20 (pH 8.0)], and bound diabodies were eluted with 5 mL elusion buffer [300 mmol/L NaCl, 250 mmol/L imidazole, 20 mmol/L HEPES (pH 8.0)] and dialyzed in endotoxin-free PBS overnight at room temperature. Samples were filtered with 0.22 μm filters and stored at −20° C. until their use. Purity of the preparation was determined by size-exclusion chromatography profile (fast protein liquid chromatography; Superdex 75™; Amersham Pharmacia Biotech) as necessary.

For preparative analysis of the diabody, purified diabody preparations were run on 4% to 20% Tris-glycine gel (Invitrogen) and bands were visualized using GelCodeTM Blue Stain Reagent (Pierce). Gels were scanned and the band intensities were analyzed using the Image J program (NIH).

### Example 4

### VEGF ELISA

The concentration of VEGF was determined using VEGF ELISA kit (R & D Systems, Minneapolis, Minn.) following the manufacturer's instructions.

### Example 5

### siRNA EMP2

EMP2 levels were decreased by transiently transfecting ARPE-19 cells with 75 picomoles EMP2 siRNA (J-016226-05 (SEQ ID NO:3), J-016226-06 (SEQ ID NO: 4), J-016226-07 (SEQ ID NO:5), J-016226-08 (SEQ ID NO:6); target sequecnces; Dharmacon, Lafayette, Colo.) and lipophilic transfection reagent (Lipofectamine™ 2000; Invitrogen, Carlsbad, Calif.) and analyzed after 48 hours. As a negative control, the cells were transfected with 75 picomoles of scramble control siRNA (D-001206-13-05; Dharmacon). The EMP2 siRNA and control siRNA are a pool of four siRNAs targeting EMP2 or a pool of four nontargeting siRNAs, respectively. The level of VEGF expression was quantified by Western blot and ELISA as described.

### Example 6

### VEGF Western Blot Analysis

Western blot analysis was performed as previously described. Briefly, cell protein was isolated by using RIPA buffer containing protease and phosphatase inhibitors (Upstate, Charlottesville, Va.) and the protein concentration determined with a protein assay (bicinchoninic acid (BCA); Bio-Rad). A total of 10 μg protein was loaded in each lane and the proteins fractionated by 4% to 20% SDS-PAGE gradient gel in reducing conditions. Proteins were transferred to nitrocellulose membranes (GE Healthcare, Buckinghamshire, UK) and the adequacy of transfer confirmed by Ponceau S red staining (Sigma-Aldrich). The membrane was then blocked with nonfat milk in TBS Tween (TBST; Upstate). Blots were incubated for 1 hour with primary antibody at a dilution of 1: for ant-VEGF and 1:5000 for β-actin. Horseradish peroxidase-conjugated goat anti-rabbit or horseradish peroxidase-conjugated goat anti-mouse was exposed to the blots at a 1:2000 dilution. The blots were then developed with chemiluminescence to visualize bound antibody (ECL; Pierce, Rockford, Ill.) and quantified with β-actin as the internal control. The Western blot analyses were quantified with NIH Image J. The blots were digitized with a flatbed scanner, and the band density was measured by using Image J. To account for loading variability, β-actin was used to normalize each sample. At least three independent experiments were performed and, where indicated, the results were evaluated for statistical significance with a Student's t-test (unpaired, one-tail). A level of P<0.05 was considered to be statistically significant.

SEQ ID NO: 1
Epithelial Membrane Protein 2 (NP_001415.1)
MLVLLAFITAFHITSAALLFIATVDNAWWVGDEFFADVWRICTNNTNCTV
INDSFQEYSTLQAVQATMILSTILCCIAFFIFVLQLFRLKQGERFVLTSI
IQLMSCLCVMIAASIYTDRREDIHDKNAKFYPVTREGSYGYSYILAWVAF
ACTFISGMMYLILRKRK
SEQ ID NO: 2
Epithelial Membrane Protein 2 (NM_001424)
GAGGGGCCCC GCCGCCTAGA GGGTGGAGGG AGGGCGCGCA
GTCCCAGCCC AGAGCTTCAA AACAGCCCGG CGGCCTCGCC
TCGCACCCCC AGCCAGTCCG TCGATCCAGC TGCCAGCGCA
GCCGCCAGCG CCGGCACATC CCGCTCTGGG CTTTAAACGT
GACCCCTCGC CTCGACTCGC CCTGCCCTGT GAAAATGTTG
GTGCTTCTTG CTTTCATCAT CGCCTTCCAC ATCACCTCTG
CAGCCTTGCT GTTCATTGCC ACCGTCGACA ATGCCTGGTG
GGTAGGAGAT GAGTTTTTTG CAGATGTCTG GAGAATATGT
ACCAACAACA CGAATTGCAC AGTCATCAAT GACAGCTTTC
AAGAGTACTC CACGCTGCAG GCGGTCCAGG CCACCATGAT
CCTCTCCACC ATTCTCTGCT GCATCGCCTT CTTCATCTTC
GTGCTCCAGC TCTTCCGCCT GAAGCAGGGA GAGAGGTTTG
TCCTAACCTC CATCATCCAG CTAATGTCAT GTCTGTGTGT
CATGATTGCG GCCTCCATTT ATACAGACAG GCGTGAAGAC
ATTCACGACA AAAACGCGAA ATTCTATCCC GTGACCAGAG
AAGGCAGCTA CGGCTACTCC TACATCCTGG CGTGGGTGGC
CTTCGCCTGC ACCTTCATCA GCGGCATGAT GTACCTGATA
CTGAGGAAGC GCAAATAGAG TTCCGGAGCT GGGTTGCTTC
TGCTGCAGTA CAGAATCCAC ATTCAGATAA CCATTTTGTA
TATAATCATT ATTTTTTGAG GTTTTTCTAG CAAACGTATT
GTTTCCTTTA AAAGCCAAAA AAAAAAAAAA AAAAAAAAAA
AAAAGAAAAA AGAAAAAAAA AATCCAAAAG AGAGAAGAGT
TTTTGCATTC TTGAGATCAG AGAATAGACT ATGAAGGCTG
GTATTCAGAA CTGCTGCCCA CTCAAAAGTC TCAACAAGAC
ACAAGCAAAA ATCCAGCAAT GCTCAAATCC AAAAGCACTC
GGCAGGACAT TTCTTAACCA TGGGGCTGTG ATGGGAGGAG
AGGAGAGGCT GGGAAAGCCG GGTCTCTGGG GACGTGCTTC
CTATGGGTTT CAGCTGGCCC AAGCCCCTCC CGAATCTCTC
TGCTAGTGGT GGGTGGAAGA GGGTGAGGTG GGGTATAGGA
GAAGAATGAC AGCTTCCTGA GAGGTTTCAC CCAAGTTCCA
AGTGAGAAGC AGGTGTAGTC CCTGGCATTC TGTCTGTATC
CAAACCAGAG CCCAGCCATC CCTCCGGTAT CGGGGTGGGT
CAGAAAAAGT CTCACCTCAA TTTGCCGACA GTGTCACCTG
CTTGCCTTAG GAATGGTCAT CCTTAACCTG CGTGCCAGAT
TTAGACTCGT CTTTAGGCAA AACCTACAGC GCCCCCCCCC
TCACCCCAGA CCTACAGAAT CAGAGTCTTC AAGGGATGGG
GCCAGGGAAT CTGCATTTCT AACGCGCTCC CTGGGCAACG
CTTCAGATGC GTTGAAGTTG GGGACCACGG TGCCTGGGCC
AGGTCAGCAG AGCTGCCTCG TAAATGCTGG GGTATCGTCA
TGTGGAGATG GGGAGGTGAA TGCAACCCCC ACAGCAGGCC
AAAACCTTGG CCTCCATCGC CACAGCTGTC TACATCTAGG
GCCCCAAAAC TCCATTCCTG AGCCATGTGA ACTCATAGAC
ACCTTCAGGG TGTGGGGTAC AGCCTCCTTC CCATCTTATC
CCAGAAGGCC TCTCCCTTCT TGTCCAGCCC TTCATGCTAC
ACCTGGCTGG CCTCTCACCC CTATTTCTAG AGCCTCAGAG
GACCCATCCA CCATTCATTC ATTCATTCAT TCATTCATTC
ATTCATTCAT TCATCAACAT AAATCATAAC TTGCATGCAT
GTGCCAGGCA CAGGGGATAC CCTCTAGAGA CAATCTCCTC
CTAGGGCTCA TGGCCTAGTG GAGGAGACAG ATTAAAACTT
AATTAGAAAA ACTGGCTGGG TACAGTGGCT CATGCTTGTA
ATCCCAGCAC TTTGGGAGGC TGAGGCGGGT GGATCACCTG
AGGTCAGGAG TTCAAGACCA GCCTGGCCAA AATGGTAAAA
CCTGTCTCTA CTAAAAATAC AAAAATGAGC TGGGCGTGGT
GGTGCATGCC TGTAATCCCA GCTATCAGGT GGCTGAGGCA
GGAGAATCAC TTGAAATGGG AGGTGGAGGT TGCAGTGAGC
CGAGACCGTG CCACTGCACT CCAGCCTGGG TGACAGAGTG
AGACTCCATC TCAAAAAAAG AAAAAAAAGA AAAGAAACTA
ATTACACACT GTGATGGAGG CTGCAAAGAA CACCACTAAG
AATTCAAAAT CAGCTGGGTG CGGTGGCTCA CACCTGTAAT
CCCAGCACTT TGGGAGGCTG AGGCAGGTGG ATCACAAGGT
CAGGAGTTCA AGACCAGCCT GGCCAACATG GTGAAACCCC
GTCTCTACCG AAAATACAAC AAAATTAGCC CGGTGTGGTG
GCAGGTGCCT GTAATCCCAG CTACTTAGGA GGCTGAGGCA
GGAGAATCGC TTGAAACTGG GAGGCGGAGG TCGCAGTGAG
CCGAGATTCA CCACTGCACT CCAGCCCAGG CGACAGTCTG
AGACTCCGTC TCAAAAATAA AACGATTCAA AATCGAGGCC
TGTGGCATGG TAGGGAGGCT GCTTTACGCG TGCCTATTAT
TAAATGCTCC TGGAGGCATT TAGGTATTTA GATCAGTCTA
AATATAGCTC CATTCAGTTC GTGCAGATGA CAGTTATTGG
GCAGTACCTG TCTGTGTAAC ACCCAGAAAA CATGTCTGTG
GAGGGGCCCA TGGTCCCGAC AGTAAATGCG GTGAGAGGGT
CCCATAGAGC TGGAGTTTTC AAGCTTTAGG GGTTCCCGTG
CTGCTTGGGA CAGGCTGATT CAGAGGGTCT GGGTGAATGA
TTTCCAGGTG ATTTTAAGAC TGTGCTGAGA AATAGGGCTT
TTGGGGCCTT GTCCTTCAGG ATCAAAGCAT GATGCTGTGT
GGCAATGCAG ACCACCCAGG AACCATCCCA GGAGATAAGC
TCTTTGCACC TCATTGTCTT TTTCTGCTTA TGTTGGAGCA
GGATGCTGGG GGCTGTCCTG GGATGGGGTG TGGGACCTCG
TGCTATTTAA ATACTTTTGC ACTTGACCTT CTGCTGAGTG
GAGTGGTGGT TTGCCATCAG CTCAGTTCCA GTGGAGCTGA
AGAGACATCT GGTTTGAGTA GTTTTAGGGC CACCATGGAT
ATCTCTTCAA TGCAGGATTG GCTCTTTCCA TCTGCTCTTT
CATTCATTTG TTTTTGACAG ATAGTATTAA ATGTTTACCA
TGTTCCAGGC ACTGTGTGAG GCTCTGAAAA TACAGGGGTG
AGCAAATCCA GATATCCTCC CTGCCATCAT GAAGTTTGGA
GTCTATGAGA TAGGACCCCC TCCCTATGGA GAAGCCACCA
ATGCAGTACA GGGTGACCTG GGGCCAGAGA CAGGACAAAT
GTCACCTCCT GCCTCCATGA GATACTCTCA CTAGTCATAT
TGTGGGCAAG AATGTGGCTT ACACCCCTAG GGTTAACAGG
ATGCTACCCA AGCTCATGGA GGAAGTTGAA TCTTAAGTTC
CCTTGAAACT TTCTACCTTG GTGGCTTTTC TATAATTTTC
TTTTTTCTTT TTCTTTTTTT TTTTTTTTTT TGAGACTGAG
TTTTGCTCTT GTTGCCCAGG CTGGAGTGCA GTGGCACCAT
CTTGGCTCAC CGCAACCTCT GCCTCCTGGG TTCAAGTGAT
TCTCCTGCCT CAGCCTCCCG AGTAGCTGGG ATTACAGGCA
TGTCCCACCA TGCCCAGCTA ATTTTTGTAT TTTTAGTAGA
GATGGGGTTT CTCCATGTTG GTCAGGCTGG TTTCGAACTC
CCAACCTCAG GTGATCCGCC CACCTCAGCC TTCCAAAGTG
CTGGGATTAC AGGCATGAGC CACTGCGTCT GGCCTTCTAT
AATTTTCTGG TAGTCACGAT GGAAACAAAC AAAACACCTT
AGAACCAGAG ATCGACCCCC TCAAGCAATA CATCAATTCC
CTTCACAAGA AACGTCGGGG CTACATGAGT ATCTGTGTTG
AATGCGGTCT GAAATGATCC TATGGATTTT CCCGGCTGGT
TGCCACTGCT GTACAACATT CAGTGCCCAC ATCCACCTGT
GCCATTAAGC TTTTTTGAGA CATGAGAGAT GCCTCTTCCC
TGCTGTATGA CATGCATTTG GGAAGTTGGA AAGAAATGAC
AAAATCAGGG AGAAAACATC CAAGCTTCTT ACCTGTAGAT
AGAATCAGCC CTCACTTGGT GCTTATTACC AGTTATTCAA
GAACAATAAC AACAACAAAA TTAGTAGACA TCCAAGAAGC
ACATATTAGG ACCAAAGATA GCATCAACTG TATTTGAAGG
AACTGTAGTT TGCGCATTTT ATGACATTTT TATAAAGTAC
TGTAATTCTT TCATTGAGGG GCTATGTGAT GGAGACAGAC
TAACTCATTT TGTTATTTGC ATTAAAATTA TTTTGGGTCT
CTGTTCAAAT GAGTTTGGAG AATGCTTGAC TTGTTGGTCT
GTGTGAATGT GTATATATAT ATACCTGAAT ACAGGAACAT
CGGAGACCTA TTCACTCCCA CACACTCTGC TATAGTTTGC
GTGCTTTTGT GGACACCCCT CATGAACAGG CTGGCGCTCT
AGGACGCTCT GTGTTCACTG ATGATGAAGA AACCTAGAAC
TCCAAGCCTG TTTGTAAACA CACTAAACAC AGTGGCCTAG
ATAGAAACTG TATCGTAGTT TAAAATCTGC CTCGCGGGAT
GTTACTAAAC TCGCTAATAG TTTAAAGGTT ACTTACAATA
GAGCAAGTTG GACAATTTTG TGGTGTTGGG GAAATGTTAG
GGCAAGGCCT AGAGGTTCAT TTTGAATCTT GGTTTGTGAC
TTTAGGGTAG TTAGAAACTT TCTACTTAAT GTACCTTTAA
AATAGTCCAT TTTCTATGTT TTGTATAATC TGAAACTGTA
CATGGAAAAT AAAGTTTAAA ACCAGATTGC CCAGAGCAAG
ACTCTAATGT TCCCAACGGT GATGACATCT AGGGCAGAAT
GCTGCCATTT TGAGGGGCAG GGGGTCAGCT GATTTCTCAT
CAAGATAATA ATGTATGGTT TTTACACTAA GCAACTGATA
AATGGACAAT TTATCACTGG AAAAAA
SEQ ID NO: 3
(Target Sequence 1): GUACCAACAACACGAAUUG
SEQ ID NO: 4
(Target Sequence 2): UGAUUGCGGCCUCCAUUUA
SEQ ID NO: 5
(Target Sequence 3): GCGAAAUUCUAUCCCGUGA
SEQ ID NO: 6
(Target Sequence 4): AGACAGGCGUGAAGACAUU
SEQ ID NO: 7
KS49 heavy chain
MAQVQLVQSGGGVVQPGRSLRLSCAASGFTFSSYAMHWVRQAPG
KGLEWVAVISYDGSNKYYADSVKGRFTISRDNSKNTLYLQMNSLR
AEDTAVYYCARDRRGRKSAGIDYWGQGTLVTVSS
SEQ ID NO: 8
KS49 heavy chain CDR1: SYAMH
SEQ ID NO: 9
KS49 heavy chain CDR2: VISYDGSNKYYADSVKG
SEQ ID NO: 10
KS49 heavy chain CDR3: DRRGRKSAGIDY
SEQ ID NO: 11
KS49 light chain
DIQMTQSPSSLSASVGDRVTITCQASQDISNYLNWYQQKPGKAPK
LLIYAASSLQSGVPSRFSGSGSGTDFTLTISSLQPEDFATYYCLQDY
NGWTFGQGTKVDIKRAAAEQKLISEEDLNGAA
SEQ ID NO: 12
KS49 light chain CDR1: QASQDISNYLN
SEQ ID NO: 13
KS49 light chain CDR2: AASSLQS
SEQ ID NO: 14
KS49light chain CDR3: LQDYNGWT
SEQ ID NO: 15
KS83 heavy chain
MAQVQLVESGGGLVQPGGSLRLSCAASGFTFSSYAMHWVRQAPG
KGLEWVAVISYDGSNKYYADSVKGRFTISRDNSKNTLYLQMNSLR
AEDTAVYYCARTVGATGAFDIWGQGTMVTVSSS
SEQ ID NO: 16
KS83 heavy chain CDR1: SYAMH
SEQ ID NO: 17
KS83 heavy chain CDR2: VISYDGSNKYYADSVKG
SEQ ID NO: 18
KS83 heavy chain CDR3: TVGATGAFDI
SEQ ID NO: 19
KS83 light chain-DIVMTQSPSTVSASVGD RV
IIPCRASQSIGKWLAWYQQKPGKAPKLLIYKASSLEGWVPSRFSGSGSGT
EFSLTISSLQPDDSATYVCQQSH NFPPTFGGGTKLEIKRAAAEQKLISE
EDLNGAA
SEQ ID NO: 20
KS83 light chain CDR1: RASQSIGKWLA
SEQ ID NO: 21
KS83 light chain CDR2: KASSLEG
SEQ ID NO: 22
KS83 light chain CDR3: QQSHNFPPT
SEQ ID NO: 23
KS41 Heavy Chain-
MAQVQLVQSGGGLVQPGRSLRLSCAASGFSFSEYPMHWVR
QAPGRGLESVAVISYDGEYQKYADSVKGRFTISRDDSKSTV
YLQMNSLRPEDTAVYYCARTINNGMDVWGQGTTVTVSS
SEQ ID NO: 24
KS41 heavy chain CDR1: EYPMH
SEQ ID NO: 25
KS41 heavy chain CDR2: VISYDGEYQKYADSVKG
SEQ ID NO: 26
KS41 heavy chain CDR3: TINNGMDV
SEQ ID NO: 27
KS41 Light Chain-
DIVMTQSPSSLSASVGDRVTITCRASQGIRNDLGWYQQKPGKAPELLIYG
ASSLQSGVPSRFSGSGSGTDFTLTISSLQPEDSATYYCLQDYNGWTFGQG
TKLEIKRAAAEQKLISEEDLNGAA
SEQ ID NO: 28
KS41 light chain CDR1: RASQGIRNDLG
SEQ ID NO: 29
KS41 light chain CDR2: GASSLQS
SEQ ID NO: 30
KS41 light chain CDR3: LQDYNGWT
SEQ ID NO: 31
KS89 Heavy Chain-
MAQVQLVQSGGGLVQPGRSLRLSCAASGFSFSEYPMHWVRQAPGRGLESV
AVISYDGEYQKYADSVKGRFTISRDDSKSTVYLQMNSLRPEDTAVYYCAR
TINNGMDVWGQGTTVTVSS
SEQ ID NO: 32
KS89 heavy chain CDR1: EYPMH
SEQ ID NO: 33
KS89 heavy chain CDR2: VISYDGEYQKYADSVKG
SEQ ID NO: 34
KS89 heavy chain CDR3: TINNGMDV
SEQ ID NO: 35
KS89 Light Chain-
DIVMTQSPSSLSASVGDRVTITCRASQGIRNDLGWYQQKPGKAPELLIYG
ASSLQSGVPSRFSGSGSGTDFTLTISSLQPEDSATYYCLQDYNGWTFGQG
TKLEIKRAAAEQKLISEEDLNGAA
SEQ ID NO: 36
KS89 light chain CDR1: RASQGIRNDLG
SEQ ID NO: 37
KS89 light chain CDR2: GASSLQS
SEQ ID NO: 38
KS89 light chain CDR3: LQDYNGWT
SEQ ID NO: 39
KS49 full polypeptide sequence of
anti-EMP-2 diabody
MAQVQLVQSGGGVVQPGRSLRLSCAASGFTFSSYAMHWVRQAPG
KGLEWVAVISYDGSNKYYADSVKGRFTISRDNSKNTLYLQMNSLRAEDTA
VYYCARDRRGRKSAGIDYWGQGTLVTVSSGGGSDIQMTQSPSSLSASVGD
RVTITCQASQDISNYLNWYQQKPGKAPKLLIYAASSLQSGVPSRFSGSGS
GTDFTLTISSLQPEDFATYYCLQDY NGWTFGQGTKVDIKRAAAEQKLIS
EEDLNGAAHHHHHH
SEQ ID NO: 40
KS83 full polypeptide sequence of
anti-EMP-2 diabody
MAQVQLVESGGGLVQPGGSLRLSCAASGFTFSSYAMHWVRQAPGKGLEWV
AVISYDGSNKYYADSVKGRFTISRDNSKNTLYLQMNSLRAEDTAVYYCAR
TVGATGAFDIWGQGTMVTVSSSGGGSDIVMTQSPSTVSASVGDRVIIPCR
ASQSIGKWLAWYQQKPGKAPKLLIYKASSLEGWVPSRFSGSGSGTEFSLT
ISSLQPDDSATYVCQQSHNFPPTFGGGTKLEIKRAAAEQKLISEEDLNGA
AHHHHHH
SEQ ID NO: 41
KS41 full polypeptide sequence of
anti-EMP-2 diabody
MAQVQLVQSGGGLVQPGRSLRLSCAASGFSFSEYPMHWVRQAPGRGLESV
AVISYDGEYQKYADSVKGRFTISRDDSKSTVYLQMNSLRPEDTAVYYCAR
TINNGMDVWGQGTTVTVSSSGGGSDIVMTQSPSSLSASVGDRVTITCRAS
QGIRNDLGWYQQKPGKAPELLIYGASSLQSGVPSRFSGSGSGTDFTLTIS
SLQPEDSATYYCLQDYNGWTFGQGTKLEIKRAAAEQKLISEEDLNGAAHH
HHHH
SEQ ID NO: 42
KS89 full polypeptide sequence of
anti-EMP-2 diabody
MAQVQLVQSGGGLVQPGRSLRLSCAASGFSFSEYPMHWVRQAPGRGLESV
AVISYDGEYQKYADSVKGRFTISRDDSKSTVYLQMNSLRPEDTAVYYCAR
TINNGMDVWGQGTTVTVSSSGGGSDIVMTQSPSSLSASVGDRVTITCRAS
QGIRNDLGWYQQKPGKAPELLIYGASSLQSGVPSRFSGSGSGTDFTLTIS
SLQPEDSATYYCLQDYNGWTFGQGTKLEIKRAAAEQKLISEEDLNGAAHH
HHHH

1. A method for treating wet age related macular degeneration (AMD), the method comprising the administration of an effective amount of an epithelial membrane protein 2 (EMP2) inhibitor to a subject in need thereof.

2. The method of claim 1, wherein the EMP2 inhibitor is an anti-EMP2 antibody.

3. The method of claim 1, wherein the EMP2 inhibitor is an EMP2 si-RNA.

4. The method according to claim 1, wherein the EMP2 inhibitor is administered by an intraocular route or topically.

5. The method according to claim 4, wherein the EMP2 inhibitor is administered by an intraocular route.

6. A method for treating a disease of the eye in a subject comprising administering to a subject in need of treatment an effective amount of an EMP2 inhibitor.

7. The method of claim 6, wherein the disease of the eye is diabetic retinopathy, corneal neovascularization, choroidal neovascularization, cyclitis, Hippel-Lindau Disease, retinopathy of prematurity, pterygium, histoplasmosis, iris neovascularization, macular edema, glaucoma-associated neovascularization, and Purtscher's retinopathy.

8. A method for reducing the expression of vascular endothelial growth factor (VEGF) in the eye, the method comprising the administration of an effective amount of an epithelial membrane protein 2 (EMP2) inhibitor to a subject in need thereof.

9. The method of claim 8, wherein the EMP2 inhibitor is an anti-EMP2 antibody.

10. The method of claim 2 or 9, wherein the anti-EMP2 antibody comprises a heavy chain and a light chain, wherein the heavy chain comprises three complementarity determining regions (CDRs): HCDR1, HCDR2, and HCDR3, and wherein the light chain comprises three CDRs: LCDR1, LCDR2, and LCDR3, wherein HCDR1 comprises SEQ ID NO:8, HCDR2 comprises SEQ ID NO:9, and HCDR3 comprises SEQ ID NO:10, and wherein LCDR1 comprises SEQ ID NO:12, LCDR2 comprises SEQ ID NO:13, and LCDR3 comprises SEQ ID NO:14.

11. The method of claim 10, wherein the antibody is a diabody.

12. The method of claim 3, wherein the EMP2 inhibitor is an EMP2 si-RNA.

13. The method of claim 12, wherein the EMP2 si-RNA comprises a target sequence wherein the target sequence comprises SEQ ID NO:3, SEQ ID NO:4, SEQ ID NO:5, or SEQ ID NO:6.

14. The method of claim 12, wherein the EMP2 si-RNA comprises a target sequence selected from the group consisting of SEQ ID NO:3, SEQ ID NO:4, SEQ ID NO:5, and SEQ ID NO:6.

15. A method for treating wet age related macular degeneration (AMD), the method comprising the administration of an effective amount of at least two epithelial membrane protein 2 (EMP2) inhibitors to a subject in need thereof.

16. The method of claim 15, wherein one of the at least two EMP2 inhibitors is an anti-EMP2 antibody and wherein one of the at least two EMP2 inhibitors is an EMP2 si-RNA.

17. The method of claim 2, wherein the anti-EMP2 antibody comprises a heavy chain and a light chain, wherein the heavy chain comprises three complementarity determining regions (CDRs): HCDR1, HCDR2, and HCDR3, and wherein the light chain comprises three CDRs: LCDR1, LCDR2, and LCDR3, wherein HCDR1 comprises SEQ ID NO:8, HCDR2 comprises SEQ ID NO:9, and HCDR3 comprises SEQ ID NO:10, and wherein LCDR1 comprises SEQ ID NO:12, LCDR2 comprises SEQ ID NO:13, and LCDR3 comprises SEQ ID NO:14.

18. The method of claim 8, wherein the EMP2 inhibitor is an EMP2 si-RNA.
